(19) United States
(12) Patent Application Publication
(10) Pub. No.: US 2024/0415422 A1
(43) Pub. Date: Dec. 19, 2024

(54) ANALYTE MONITORING DEVICES HAVING A STRAIN GAUGE

(71) Applicant: Abbott Diabetes Care Inc., Alameda, CA (US)

(72) Inventor: Matt WALDRON, Hickory, NC (US)

(21) Appl. No.: 18/744,208

(22) Filed: Jun. 14, 2024

Related U.S. Application Data
(60) Provisional application No. 63/508,759, filed on Jun. 16, 2023.

(51) Int. Cl. A61B 5/145; A61B 5/00

(52) U.S. Cl. CPC A61B 5/14532; A61B 5/0002; A61B 5/14503; A61B 5/746; A61B 2562/0261

(57) ABSTRACT

A glucose monitoring device includes a housing configured to be placed on a skin surface of a user. The glucose monitoring device includes sensor electronics arranged within the housing. The glucose monitoring device further includes a glucose sensor that includes a first portion configured to be placed under the skin surface of the user for measuring signals indicative of glucose in a bodily fluid and a second portion configured to be arranged above the skin surface and that is coupled to the sensor electronics. The glucose monitoring device further includes a strain gauge disposed on the first portion and configured to detect a strain applied to the first portion.

### FIELD

Embodiments described herein relate to analyte monitoring devices for detecting analyte levels in a bodily fluid and having a strain gauge for measuring strain applied to the analyte sensor.

### BACKGROUND

Analyte monitoring devices can be used to detect the presence of and measure the concentration of one or more analytes in a bodily fluid. Analyte monitoring devices may be helpful to track the health of an individual or to monitor a disease state. The analyte data collected by the analyte monitoring device may be used to help the user to avoid complications associated with extreme analyte levels, such as hypoglycemia or hyperglycemia for diabetic individuals. The analyte data may also be used to help make treatment decisions, such as whether and when to administer medication and what dosage of medication to administer. The analyte data may also be used to help the individual make behavioral and lifestyle changes, such as to determine how certain foods or activities impact the user's analyte levels.

### SUMMARY OF THE INVENTION

Some embodiments described herein relate to an analyte monitoring device that includes a housing, sensor electronics arranged within the housing, and an analyte sensor. The analyte sensor includes a first portion configured to be placed under the skin surface of the user for measuring signals indicative of an analyte in a bodily fluid and a second portion configured to be arranged above the skin surface and that is coupled to the sensor electronics. A strain gauge is disposed on the first portion and is configured to detect a strain on the first portion.

In any of the various embodiments described herein, the strain gauge may be disposed on a first side of the first portion, and a second strain gauge may be disposed on a second side of the first portion opposite the first side of the first portion.

In any of the various embodiments described herein, the strain gauge may include a strain-sensitive resistor. In some embodiments, the analyte monitoring device may further include a detection circuit comprising a voltage input, a voltage output, and the strain-sensitive resistor. In some embodiments, the sensor electronics may be configured to apply a drive voltage to the detection circuit and to detect a voltage output of the detection circuit.

In any of the various embodiments described herein, the strain gauge may include a piezoresistive trace.

In any of the various embodiments described herein, the first portion may include a sensing region having one or more electrodes and an analyte-responsive reagent.

In any of the various embodiments described herein, at least a portion of the strain gauge may be encapsulated by a membrane. Optionally, the piezoresistive trace of the strain gauge may be encapsulated by the membrane.

Some embodiments described herein relate to an analyte monitoring system that includes an analyte monitoring device having an analyte sensor with a first portion configured to be placed under a skin surface of a user to detect signals indicative of analyte levels in a bodily fluid and a second portion coupled to sensor electronics. The analyte monitoring device further includes a strain gauge disposed on the first portion and configured to detect a strain applied to the first portion. The analyte monitoring system further includes one or more processors in communication with the analyte monitoring device, and configured to determine an analyte level based on the signals detected by the analyte sensor, determine a strain applied to the first portion detected by the strain gauge, and provide an analyte level alarm based on the analyte level and the strain detected by the strain gauge.

In any of the various embodiments described herein, the analyte level alarm may be output when the analyte level crosses an analyte level threshold and no strain is detected by the strain gauge. The reference to “crosses an analyte level threshold” herein may refer to an analyte level exceeding an analyte level threshold or falling below an analyte level threshold. For example, the reference to “crosses an analyte level threshold” may refer to an analyte level exceeding a high analyte level threshold or an analyte level falling below a low analyte level threshold.

In any of the various embodiments described herein, the analyte level alarm may be output when the analyte level crosses an analyte level threshold and the strain detected by the strain gauge is below a predetermined level. For example, the analyte level alarm may be output when the analyte level exceeds an analyte level threshold and the strain detected by the strain gauge is below a predetermined level.

In any of the various embodiments described herein, the analyte monitoring system may further include a receiver device in communication with the analyte monitoring device. In some embodiments, the receiver device includes the one or more processors of the analyte monitoring system such that the receiver device is configured to provide the analyte level alarm.

In any of the various embodiments described herein, the analyte level alarm may include a first alarm condition, and the analyte level alarm may be adjusted to a second alarm condition when strain is detected by the strain gauge. The analyte level alarm may be adjusted, i.e. changed, from the first alarm condition to the second alarm condition when strain is detected by the strain gauge. The analyte level alarm may be output when the relevant alarm condition is satisfied. The relevant alarm condition may be the first alarm condition. The relevant alarm condition may be changed from the first alarm condition to the second alarm condition when strain is detected by the strain gauge. In some embodiments, the first alarm condition may include a first analyte level threshold, and the second alarm condition may include a second analyte level threshold that is different than the first analyte level threshold. In some embodiments, the first alarm condition may be satisfied when a first number of analyte levels crosses an analyte level threshold, and the second alarm condition may satisfied when a second, greater number of analyte levels crosses the analyte level threshold. In some embodiments, the first alarm condition is satisfied when an analyte level crosses, optionally exceeds, an analyte level threshold, wherein the second alarm condition is satisfied when the analyte level is above (i.e. exceeds) the analyte level threshold for a predetermined period of time. In some embodiments, the first alarm condition is satisfied when an analyte level crosses, optionally falls below an analyte level threshold, wherein the second alarm condition is satisfied when the analyte level is below the analyte level threshold for a predetermined period of time.

Some embodiments described herein relate to a method of determining successful insertion of an analyte sensor that includes detecting a strain applied to an analyte sensor by a strain gauge disposed on the analyte sensor and determining successful sensor insertion when no strain is detected after insertion of the sensor under the skin surface of the user, or determining unsuccessful sensor insertion when a strain is detected after insertion of the sensor under the skin surface of the user. The method further includes transmitting an indication of the successful sensor insertion or unsuccessful sensor insertion to a receiver device in wireless communication with the analyte sensor, and outputting, by the receiver device, a notification indicative of successful sensor insertion or unsuccessful sensor insertion based on the indication.

In any of the various embodiments described herein, the strain gauge may include a detection circuit comprising a strain-sensitive resistor, and a method of determining successful sensor insertion may further include applying a voltage to the detection circuit and detecting an output voltage, wherein a strain is detected when the output voltage is non-zero. In some embodiments, the strain gauge may include a piczoresistor.

The above description of the strain gauge and analyte sensor equally apply to this method of determining successful sensor insertion. The method of determining successful sensor insertion may be performed using the analyte monitoring devices or analyte monitoring systems described herein.

Some embodiments described herein relate to an analyte monitoring device that includes a housing, sensor electronics arranged within the housing, and an analyte sensor at least partially arranged within the housing and coupled to the sensor electronics. The analyte sensor includes a first portion comprising a sensing region configured to be placed under a skin surface of a user, and a second portion configured to be placed above the skin surface and having one or more electrical contacts for communication with the sensor electronics. The analyte monitoring device further includes a strain gauge arranged on the first portion of the analyte sensor and electrically connected to an electrical contact of the one or more electrical contacts on the second portion of the analyte sensor by an electrical trace.

In any of the various embodiments described herein, the sensing region may include one or more electrodes arranged on a substrate of the analyte sensor.

In any of the various embodiments described herein, the sensing region may include a first sensing area for detecting a first analyte and a second sensing area for detecting a second analyte.

In any of the various embodiments described herein, the strain gauge may be arranged on the first portion of the analyte sensor outside of the sensing region.

In any of the various embodiments described herein, the analyte sensor may further include a substrate, a working electrode arranged on a first surface of the substrate and a counter electrode arranged on a second surface of the substrate, wherein the first and second surfaces are arranged on opposite sides of the substrate. In some embodiments, the analyte sensor further includes a dielectric layer disposed on the working electrode, and a reference electrode disposed on the dielectric layer such that the reference electrode is electrically isolated from the working electrode. In some embodiments, a second dielectric layer may be arranged on the reference electrode, and the strain gauge may be arranged on the second dielectric layer. In some embodiments, the analyte sensor may further include a dielectric layer arranged on the counter electrode, wherein the strain gauge is arranged on the dielectric layer.

In any of the various embodiments described herein, a membrane may overcoat the sensing region of the analyte sensor.

Some embodiments described herein relate to a method of providing an analyte level alarm. The method includes determining, by an analyte monitoring device that includes sensor electronics coupled to an analyte sensor, an analyte level based on the signals detected by the analyte sensor; detecting, by a strain gauge coupled to the sensor electronics, a strain applied to the first portion; and providing an analyte level alarm when an alarm condition is satisfied, wherein the alarm condition is based on the analyte level and the strain detected by the strain gauge.

In any of the various embodiments described herein, the analyte level alarm may be provided when the analyte level crosses an analyte level threshold and no strain is detected by the strain gauge.

In any of the various embodiments described herein, the analyte level alarm may be provided when the analyte level crosses an analyte level threshold and the strain detected by the strain gauge is below a predetermined level.

In any of the various embodiments described herein, the method may further include communicating data from the analyte monitoring device to a receiver device, wherein the receiver device provides the analyte level alarm.

In any of the various embodiments described herein, the method may further include adjusting the alarm condition from a first alarm condition to a second alarm condition when strain is detected by the strain gauge. In some embodiments, the first alarm condition includes a first analyte level threshold, and the second alarm condition includes a second analyte level threshold that is different than the first analyte level threshold. In some embodiments, the first alarm condition is satisfied when a first number of analyte levels crosses an analyte level threshold, the second alarm condition is satisfied when a second number of analyte levels cross the analyte level threshold, and the second number is greater than the first number. In some embodiments, the first alarm condition is satisfied when an analyte level crosses an analyte level threshold, and the second alarm condition is satisfied when the analyte level exceeds the analyte level threshold for a predetermined period of time. In some embodiments, the first alarm condition is satisfied when an analyte level crosses an analyte level threshold, and the second alarm condition is satisfied when the analyte level is below (or above) the analyte level threshold for a predetermined period of time.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows an analyte monitoring system including an analyte monitoring device according to an embodiment.

FIGS. 2A and 2B show block diagrams depicting exemplary embodiments of analyte sensors and sensor electronics according to embodiments.

FIG. 3 shows a block diagram depicting an example embodiment of a receiver device.

FIG. 4 shows a schematic view of an analyte monitoring device having a strain gauge according to an embodiment.

FIG. 5 shows a strain gauge including a piezoresistor according to an embodiment.

FIG. 6 shows an exploded view of a portion of an analyte sensor having a piezoresistor according to an embodiment.

FIG. 7 shows a front view of an analyte sensor having a strain gauge according to an embodiment.

FIG. 8 shows a cross sectional view of a portion of the analyte sensor having a strain gauge of FIG. 7.

FIG. 9 shows a front view of an analyte sensor having a strain gauge according to an embodiment.

FIGS. 10A and 10B show schematic views of an analyte sensor having a strain gauge according to embodiments.

FIG. 11 shows a diagram of a detection circuit having a strain-sensitive resistor according to an embodiment.

FIG. 12 shows a schematic view of an analyte monitoring device having multiple strain gauges according to an embodiment.

FIG. 13 shows a diagram of a detection circuit having multiple strain-sensitive resistors according to an embodiment.

FIGS. 14A and 14B show cross sectional views of a portion of an analyte monitoring device having two strain gauges in a resting orientation and in a strained orientation, respectively.

FIG. 15 shows a flowchart for providing an analyte level alarm according to an embodiment.

FIG. 16 shows a flowchart for providing an analyte level alarm according to an embodiment.

FIG. 17 shows a flowchart for determining successful or unsuccessful sensor insertion according to an embodiment.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the claims.

Analyte monitoring devices are worn on a body of a user and include a portion that is inserted under the skin surface to measure the analyte level in a bodily fluid. Analyte monitoring devices may be worn on various parts of the user's body, such as the abdomen, arm, or buttocks, among others. Analyte monitoring devices may be fully implanted under the skin of the user, or may be partially implanted, such that a first portion of the analyte sensor is inserted below the skin surface of the user to contact a bodily fluid and a second portion of the analyte sensor is arranged above the skin surface. Analyte monitoring devices may monitor analyte levels on a substantially continuous basis, such as every minute, or may monitor analyte levels on a periodic basis. The analyte data may be transmitted to a receiver device in wireless communication with the analyte monitoring device for display of the analyte data and analyte metrics to the user and/or to caregivers and healthcare professionals and to provide analyte level alarms to alert the user to potentially dangerous conditions and allow the user to take corrective action.

As the analyte data may be used to inform treatment decisions, it is important to ensure that the analyte data obtained by the analyte monitoring device is accurate and reliable. However, various sources of error may cause the analyte level determined by the analyte monitoring device to differ from the true analyte level. The ability for the analyte monitoring device to detect errors and issues that may impact the analyte level and to either correct for such errors or to notify the user is important to ensure that the user docs not make treatment decisions based on inaccurate information.

The performance of an analyte monitoring device, and ability of the analyte monitoring device to accurately determine analyte levels, may be impaired when pressure or strain is applied to the analyte sensor or to the user's body in the area of the analyte sensor. For example, when the user is seated in a couch or chair, or is lying down on a bed, pressure may be applied to the user's body in the area of the analyte monitoring device. The pressure may cause a bending or rotation of the analyte sensor that results in strain of the analyte sensor inserted under the user's skin. The pressure may result in inaccurate analyte level measurements. While it is not intended to limit the disclosure to the particular mechanism by which the analyte level measurement error is introduced, inaccurate analyte level measurements may be the result of decreased blood flow to the area of the body to which pressure is applied, mechanical impairment of the analyte sensor's operation resulting from the bending of the analyte sensor, or a combination thereof. As a result of the inaccurate analyte levels, analyte level alarms may also be inaccurate and the user may be falsely notified of a high or low analyte level, e.g., hyperglycemia or hypoglycemia, which may cause the user to take actions to address the erroneously detected condition.

Some embodiments described herein relate to an analyte monitoring device that includes an analyte sensor having a first portion configured to be arranged under the skin and in contact with a bodily fluid for monitoring an analyte level and a strain gauge for monitoring a strain on the first portion. The analyte monitoring device may use the measured/detected strain to adjust or correct the analyte level determined based on signals from the analyte sensor. The analyte monitoring device may not calculate or display an analyte level when strain is detected (or when strain above a predetermined level is detected), or the analyte monitoring device may display an analyte level along with a notification that the analyte level may be inaccurate or that sensor strain is detected. The analyte monitoring device may not provide analyte level alarms when strain is detected (or when strain above a predetermined level is detected), or the conditions for triggering an analyte level alarm may be modified when strain is detected.

The term “analyte” as used herein, may refer to, for example, glucose, ketones, lactate, oxygen, hemoglobin A1C, albumin, alcohols (e.g., ethanol), alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

The term “bodily fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears or the like. In certain embodiments, the bodily fluid is interstitial fluid.

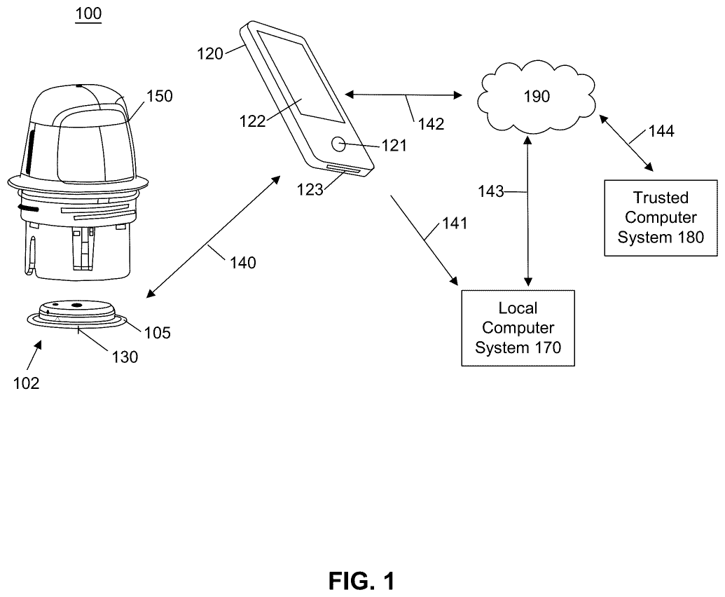

An analyte sensor system according to an embodiment is shown for example in FIG. 1. Analyte monitoring devices having strain gauges described herein may be part of an analyte monitoring system 100, as shown for example in FIG. 1. Analyte monitoring system 100 includes an analyte monitoring device 102 which may include sensor electronics electrically coupled to an analyte sensor 130 and an adhesive layer 105 for attachment to a skin surface of the user. An applicator 150 can be operated to position a portion of analyte sensor 130 through the skin surface and into fluid contact with a bodily fluid. In some embodiments, applicator 150 may position sensor electronics and/or adhesive layer 105 on the skin surface. In some embodiments, sensor electronics, analyte sensor 130 and adhesive layer 105 may be sealed within applicator 150 before use.

Analyte monitoring device 102 can communicate with a receiver device 120 via a communication path 140 using a wired or wireless technique. Example wireless communication protocols include Bluetooth, Bluetooth Low Energy (BLE, BTLE, Bluetooth SMART, etc.), Near Field Communication (NFC), and RFID, among others. Communication may be uni-directional or bi-directional. Analyte monitoring device 102 may communicate analyte data among other information regarding operation of the analyte monitoring device 102, such as error information or information associated with a sensor status.

In some embodiments, receiver device 120 may be a dedicated handheld receiver, a smartphone, PDA, laptop, smartwatch, or other mobile electronic device or wearable electronic device. Receiver device 120 may include a display 122 for outputting analyte data, alarms, and other information to the user. Receiver device 120 may include an input component 121, such as buttons, keys, a touch screen, or the like for receiving user input, such as to receive data or commands, or otherwise to control operation of receiver device 120. Receiver device 120 may include a power source, such as one or more batteries, and in some embodiments power source may be recharged via a port 123. Receiver device 120 may include one or more processors, and memory coupled to the one or more processors. Receiver device 120 may include software or programming for communicating with analyte monitoring device 102, and for processing and displaying analyte data and alarms. Receiver device 120 may be provided by a party other than the manufacturer of analyte monitoring device 102.

Receiver device 120 may communicate with a local computer system 170 via a communication path 141 using a wired or wireless technique. Local computer system 170 can include one or more of a laptop, desktop, tablet, smartphone, set-top box, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how receiver device 120 can communicate via a communications path 142 with network 190, by wired or wireless technique as described previously. Network 190 can be any of a number of networks, such as private networks and public networks, local area or wide area networks, and so forth. A trusted computer system 180 can include one or more servers (e.g., a cloud) and can provide authentication services and secured data storage and can communicate via communications path 144 with network 190 by wired or wireless communication protocols.

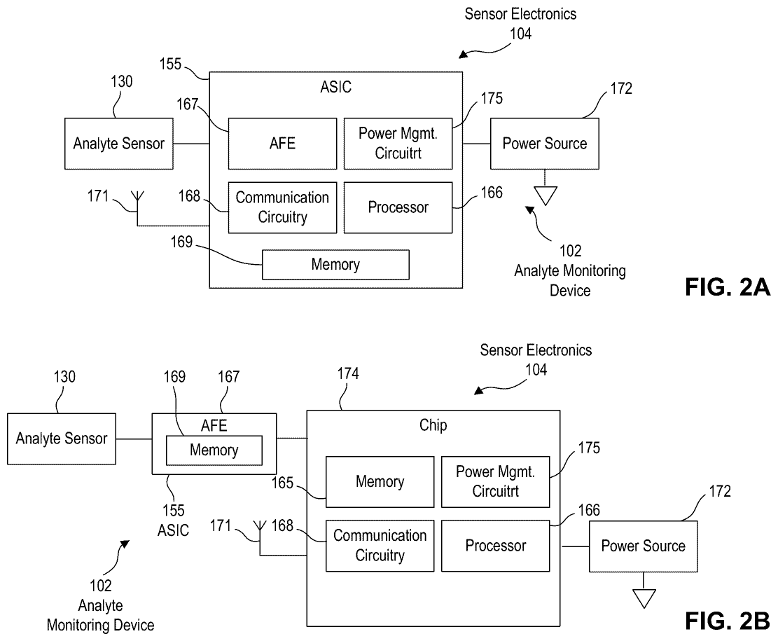

FIGS. 2A and 2B are block diagrams depicting example embodiments of analyte monitoring devices 102 having analyte sensors 130 and sensor electronics 104. In some embodiments, sensor electronics 104 (including analyte monitoring circuitry) that may have the majority of the processing capability for rendering end-result data suitable for display to the user. In FIG. 2A, a single semiconductor chip 155 is depicted that can be a custom application specific integrated circuit (ASIC). Shown within ASIC 155 are certain high-level functional units, including an analog front end (AFE) 167, power management (or control) circuitry 175, processor 166, and communication circuitry 168 (which can be implemented as a transmitter, receiver, transceiver, passive circuit, or otherwise according to the communication protocol). In this embodiment, both AFE 167 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 169 is also included within ASIC 155 and can be shared by the various functional units present within ASIC 155, or can be distributed amongst two or more of them. Memory 169 can also be a separate chip. Memory 169 can be volatile and/or non-volatile memory. In this embodiment, ASIC 155 is coupled with power source 172, which can be a coin cell battery, or the like. AFE 167 interfaces with in vivo analyte sensor 130 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to a receiver device as described herein, for example, where minimal further processing is needed by the resident software application to display the data.

FIG. 2B is similar to FIG. 2A but instead includes two discrete semiconductor chips 167 and 174, which can be packaged together or separately. In FIG. 2B, AFE 167 is resident on ASIC 155. Processor 166 is integrated with power management circuitry 175 and communication circuitry 168 on chip 174. AFE 167 includes memory 169 and chip 174 includes memory 165, which can be isolated or distributed within. In one example embodiment, AFE 167 is combined with power management circuitry 175 and processor 166 on one chip, while communication circuitry 168 is on a separate chip. In another example embodiment, both AFE 167 and communication circuitry 168 are on one chip, and processor 166 and power management circuitry 175 are on another chip. It should be noted that other chip combinations are possible, including three or more chips, each bearing responsibility for the separate functions described, or sharing one or more functions for fail-safe redundancy.

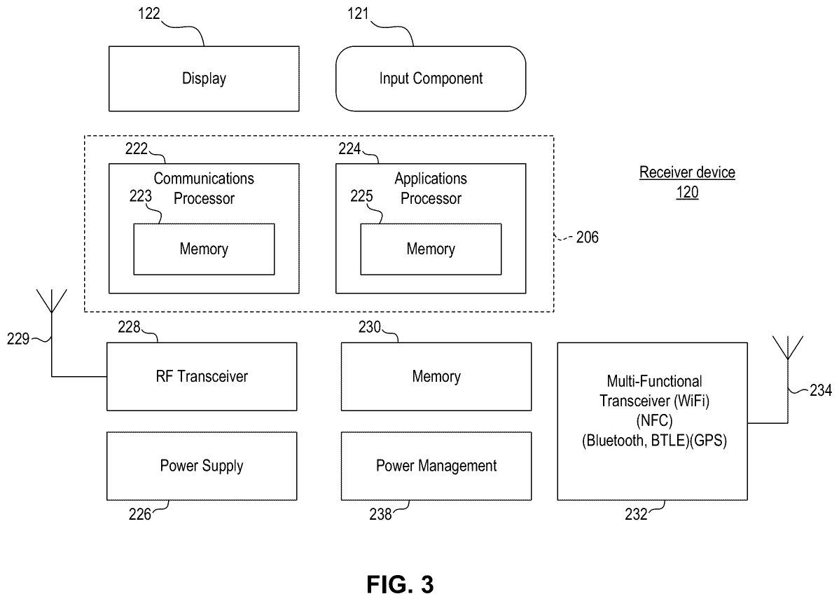

A block diagram depicting an example embodiment of a receiver device 120 in an embodiment is shown in FIG. 3. Receiver device 120 can include a display 122 and an input component 121, such as one or more keys, buttons, or a touch screen, among others. Receiver device 120 may include one or more processors coupled to memory. Receiver device 120 may include a processing core 206 including one or both of a communications processor 222 coupled with memory 223 or an applications processor 224 coupled with memory 225. Also included can be separate memory 230, RF transceiver 228 with antenna 229, and power supply 226 with power management module 238. Further, receiver device 120 can also include a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device. In some embodiments, receiver device 120 may include, for example, a dedicated handheld receiver, a smartphone, a PDA, a laptop, a watch, or other mobile electronic device or wearable electronic device.

Information may be communicated from analyte monitoring device 102 to receiver device 120 automatically and/or continuously when the analyte information is available, or may not be communicated automatically and/or continuously, but rather stored or logged in a memory of analyte monitoring device 102, e.g., for later output.

Data can be sent from sensor electronics 104 to receiver device 120 at the initiative of either analyte monitoring device 102 or receiver device 120. For example, in many example embodiments sensor electronics 104 of analyte monitoring device 102 can communicate data periodically in an unprompted or broadcast-type fashion, such that an eligible receiver device 120, if in range and in a listening state, can receive the communicated data (e.g., sensed analyte data). This is at the initiative of sensor electronics 104 because receiver device 120 does not have to send a request or other transmission that first prompts sensor electronics 104 to communicate. Broadcasts can be performed, for example, using an active Wi-Fi, Bluetooth, or BTLE connection, among others. The broadcasts can occur according to a schedule that is programmed within sensor electronics (e.g., about every 1 minute, about every 5 minutes, about every 10 minutes, or the like). Broadcasts can also occur in a random or pseudorandom fashion, such as whenever sensor electronics 104 detect a change in the sensed analyte data. Further, broadcasts can occur in a repeated fashion regardless of whether each broadcast is actually received by a receiver device 120.

Analyte monitoring system 100 can also be configured such that receiver device 120 sends a transmission that prompts analyte monitoring device 102 to communicate its data to receiver device 120. This is generally referred to as “on-demand” data transfer. An on-demand data transfer can be initiated based on a schedule stored in a memory of receiver device 120, or at the behest of the user via a user interface of receiver device 120. For example, if the user wants to check his or her analyte level, the user could perform a scan of sensor electronics 104 using a near-field communication (NFC), Bluetooth, BTLE, or Wi-Fi connection. Data exchange can be accomplished using broadcasts only, on-demand transfers only, or any combination thereof.

Once analyte monitoring device 102 is placed on the body so that at least a portion of sensor 130 is in contact with the bodily fluid and electrically coupled to the electronics within analyte monitoring device 102, sensor derived analyte information may be communicated in on-demand or unprompted (broadcast) fashion from the sensor electronics 104 to a receiver device 120. On-demand transfer can occur by first powering on receiver device 120 (or it may be continually powered) and executing a software algorithm stored in and accessed from a memory of receiver device 120 to generate one or more requests, commands, control signals, or data packets to send to sensor electronics 104. The software algorithm executed under, for example, the control of processing hardware of receiver device 120 may include routines to detect the position of the analyte monitoring device 102 relative to receiver device 120 to initiate the transmission of the generated request command, control signal and/or data packet.

Different types and/or forms and/or amounts of information may be sent as part of each on-demand or other transmission including, but not limited to, one or more of current analyte level information (i.e., real time or the most recently obtained analyte level information temporally corresponding to the time the reading is initiated), rate of change of an analyte over a predetermined time period, rate of the rate of change of an analyte (acceleration in the rate of change), or historical analyte information corresponding to analyte information obtained prior to a given reading and stored in a memory of the analyte monitoring device 102.

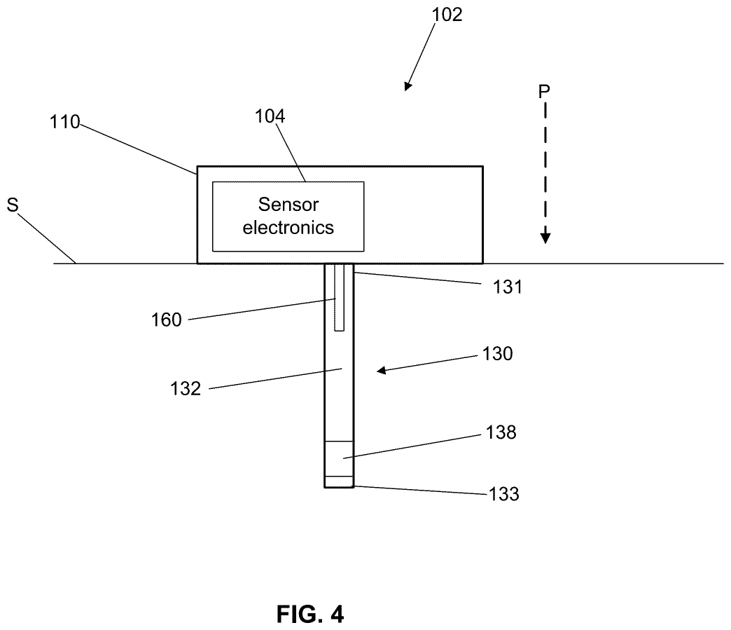

Some embodiments described herein relate to an analyte monitoring device having one or more strain gauges, as shown for example in FIG. 4. Analyte monitoring device 102 may include a housing 110 in which sensor electronics 104 and at least a portion of an analyte sensor 130 are arranged. Analyte sensor 130 is partially disposed within housing 110 and is electrically coupled to sensor electronics 104 so that signals indicative of analyte levels detected by analyte sensor 130 are communicated to sensor electronics 104. Housing 110 is configured to be placed on a skin surface of the user, and may be secured thereto via an adhesive, such that analyte monitoring device 102 is a wearable device.

Analyte sensor 130 is in electrical communication with sensor electronics 104 such that signals detected by analyte sensor 130 are communicated to sensor electronics 104. Sensor electronics 104 may process signals from analyte sensor 130 to determine an analyte data, such as analyte levels. Sensor electronics 104 may be in wireless communication with one or more receiver devices configured to display analyte data and alarms to the user as discussed herein.

In some embodiments, sensor electronics 104 may be removably coupled to housing 110. Sensor electronics 104 may be coupled to housing 110 and to analyte sensor 130 upon installation of analyte monitoring device 102 on the body of the user, such as during use of an applicator (e.g., applicator 150 in FIG. 1). However, in some embodiments, sensor electronics 104 may be permanently fixed to housing 110. Sensor electronics 104 may include a printed circuit board assembly including one or more processors, and permanent and/or volatile memory coupled to the one or more processors. Memory may store instructions for determining analyte levels and analyte metrics based on signals collected by the analyte sensor. Memory may store analyte data collected by the analyte sensor for transmission to a receiver device. Sensor electronics 104 may include a power source, such as one or more batteries. In some embodiments, sensor electronics 104 may be configured to be powered by an external power source. Sensor electronics 104 may include wireless communication circuitry, such as an antenna or transceiver, and may communicate with one or more receiving devices via wireless communication protocols, such as by RFID, NFC, or Bluetooth communication protocols, among others. Sensor electronics 104 may include additional sensors, such as a temperature sensor for detecting an ambient temperature, a skin temperature, or to estimate a temperature at the sensing region. Additional sensors may include an accelerometer or gyroscope to determine movement, speed, acceleration, a position, or an orientation of the analyte monitoring device 102.

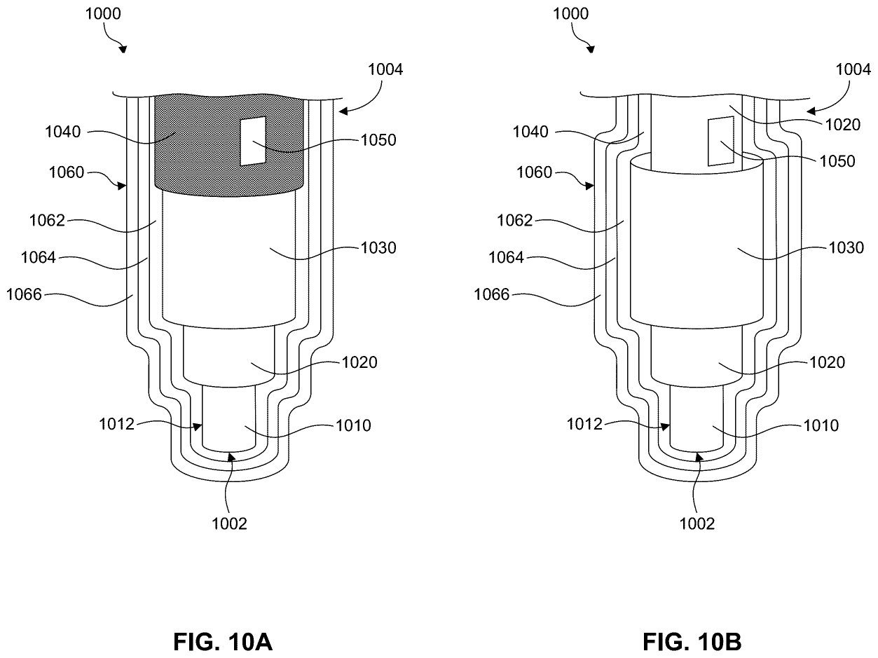

Analyte sensor 130 includes a first portion 132 extending from housing 110 and that is configured to be placed under a skin surface S to measure signals indicative of an analyte in a bodily fluid of the user. First portion 132 may be elongated and have a proximal end 131 at housing 110 and a distal end 133 opposite proximal end 131 and that is remote from housing 110. First portion 132 of analyte sensor 130 may be substantially linear in a resting position (i.e., with no strain exerted on analyte sensor 130). First portion 132 may have a planar configuration. In some embodiments, however, first portion 132 may include a substantially cylindrical configuration and may be shaped as a wire (see, e.g., FIGS. 10A and 10B).

First portion 132 of analyte sensor 130 includes one or more sensing regions 138 for monitoring one or more analytes in the bodily fluid. Sensing regions 138 may include one or more electrodes and reagents, such as an analyte-responsive reagent, which may be an analyte-responsive enzyme, for detecting the analyte in the bodily fluid. In some embodiments, sensing regions 138 may include a redox mediator, such as an osmium or ruthenium-containing complex. Sensing region 138 may include a working electrode, and may further include a counter electrode or reference electrode. Electrodes may be separated by an electrically insulating material such as a dielectric material. In embodiments in which first portion 132 has a planar configuration, electrodes may be arranged on a first side of substrate, or one or more electrodes may be placed on the first side and one or more electrodes may be placed on an opposing second side of substrate. In embodiments in which analyte sensor 130 has a cylindrical configuration, electrodes may be arranged concentrically. Sensing region 138 may further include one or more membranes disposed thereon to form a membrane assembly. Membranes may include polymeric membranes. Membranes may include a flux limiting membrane (mass transport limiting membrane or resistance membrane), a biocompatible membrane, or an interferent limiting membrane, among others. Sensing region 138 may include a sensing layer. Sensing layer may be part of membrane assembly.

In some embodiments, analyte sensor 130 may include an analyte-responsive enzyme to provide a sensing element. Some analytes, such as oxygen, can be directly electrooxidized or electroreduced at least on a working electrode of analyte sensor 130. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing element proximate to or on a surface of a working electrode. In many embodiments, a sensing element is formed near or on only a small portion of at least a working electrode.

Each sensing element includes one or more components constructed to facilitate the electrochemical oxidation or reduction of the analyte. The sensing element may include, for example, a catalyst to catalyze a reaction of the analyte and produce a response at the working electrode, an electron transfer agent to transfer electrons between the analyte and the working electrode (or other component), or both.

A variety of different sensing element configurations may be used. In certain embodiments, the sensing elements are deposited on the conductive material of a working electrode. The sensing elements may extend beyond the conductive material of the working electrode. In some cases, the sensing elements may also extend over other electrodes, e.g., over the counter electrode and/or reference electrode (or counter/reference where provided). In other embodiments, the sensing elements are contained on the working electrode, such that the sensing elements do not extend beyond the conductive material of the working electrode. In some embodiments a working electrode is configured to include a plurality of spatially distinct sensing elements. Additional information related to the use of spatially distinct sensing elements can be found in U.S. Pat. No. 10,327,677, which was filed on Dec. 8, 2011, and which is incorporated by reference herein in its entirety and for all purposes.

Sensing elements that are in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having sensing elements which contain a catalyst, including glucose oxidase, glucose dehydrogenase, lactate oxidase, or laccase, respectively, and an electron transfer agent that facilitates the electrooxidation of the glucose, lactate, or oxygen, respectively.

In some embodiments, the sensing elements are not deposited directly on the working electrode. Instead, the sensing elements may be spaced apart from the working electrode, and separated from the working electrode, e.g., by a separation layer. A separation layer may include one or more membranes or films or a physical distance. In addition to separating the working electrode from the sensing elements, the separation layer may also act as one or more of a mass transport limiting layer, an interferent eliminating layer, or a biocompatible layer.

In embodiments that include more than one working electrode, one or more of the working electrodes may not have corresponding sensing elements, or may have sensing elements that do not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing elements by, for example, subtracting the signal.

In some embodiments, the sensing elements include one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053 and 7,754,093, the disclosures of each of which are incorporated herein by reference in their entireties.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation.

Another example of an ionically-bound mediator is a positively charged polymer including quaternized poly(4-vinyl pyridine) or poly(l-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(l-vinyl imidazole) or poly(4-vinyl pyridinc).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(l-vinyl imidazole) include acrylonitrile, acrylamide, and substituted or quaternized N-vinyl imidazole, e.g., electron transfer agents with osmium complexed to a polymer or copolymer of poly(l-vinyl imidazole).

In some embodiments, electron transfer agents may have a redox potential ranging from about-200 mV to about +200 mV versus the standard calomel electrode (SCE). The sensing elements may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor works at a low oxidizing potential, e.g., a potential of about +40 mV vs. Ag/AgCl. These sensing elements use, for example, an osmium (Os)-based mediator constructed for low potential operation. Accordingly, in certain embodiments the sensing elements are redox active components that include: (1) osmium-based mediator molecules that include (bidente) ligands, and (2) glucose oxidase enzyme molecules. These two constituents are combined together in the sensing elements of the sensor.

A resistance layer or mass transport limiting layer, e.g., an analyte flux modulating layer, may be included with the sensor to act as a diffusion-limiting barrier to reduce the rate of mass transport of the analyte into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc. A mass transport limiting layer may be applied to an analyte sensor as described via any of a variety of suitable methods, including, e.g., dip coating and slot die coating, among others as would be understood by persons skilled in the art.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

In some embodiments, a membrane may be formed in situ by applying an alcohol-buffer solution of a crosslinker and a modified polymer over the enzyme-containing sensing elements and allowing the solution to cure for a period of time, such as about one to two days. The crosslinker-polymer solution may be applied over the sensing elements by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. In order to coat the distal and side edges of the sensor, the membrane material may have to be applied subsequent to singulation of the sensor precursors. In some embodiments, the analyte sensor is dip-coated following singulation to apply one or more membranes. Alternatively, the analyte sensor could be slot-die coated wherein each side of the analyte sensor is coated separately. A membrane applied in the above manner may have any combination of the following functions: (1) mass transport limitation, i.e., reduction of the flux of analyte that can reach the sensing elements, (2) biocompatibility enhancement, or (3) interferent reduction.

In some embodiments, a membrane composition for use as a mass transport limiting layer may include one or more leveling agents, e.g., polydimethylsiloxane (PDMS). Additional information with respect to the use of leveling agents can be found, for example, in U.S. Pat. No. 8,983,568, filed Sep. 30, 2008 and entitled “Analyte Sensors Comprising Leveling Agents,” the disclosure of which is incorporated by reference herein in its entirety.

In some instances, the membrane may form one or more bonds with the sensing elements. The term “bonds” is intended to cover any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing elements. In certain embodiments, crosslinking of the membrane to the sensing element facilitates a reduction in the occurrence of delamination of the membrane from the sensor.

In some embodiments, analyte sensor 130 may be factory calibrated and manufactured with minimal sensor-to-sensor variation. Calibration parameters from the factory calibration can be stored in the analyte monitoring device 102 to allow for algorithmic correction to the measured analyte data. Analyte sensor 130 can be worn for up to 7 days, up to 10 days, up to 14 days, or up to 30 days, without the need for any user calibration. This feature differs from other existing sensors which may require multiple fingerstick capillary blood glucose (BG) measurements during the wear period for calibration.

As shown in FIG. 4, analyte monitoring device 102 includes a strain gauge 160 configured to detect bending of first portion 132 of analyte sensor 130. Strain gauge 160 may detect a strain applied to first portion 132 of analyte sensor 130 as bending of first portion 132 of analyte sensor 130 may result from application of pressure P on skin surface S. The pressure P may be exerted directly on skin surface S, in a direction perpendicular to skin surface S, or may be applied at an angle, or a shear force may be applied in a direction parallel to the skin surface S, such as tugging or pulling on the skin. Such forces may be applied for example, when the user is seated or laying down on a piece of furniture, or otherwise when a person or object is pressing the skin of the user in the area of the analyte monitoring device 102.

Strain gauge 160 may be arranged on a region of first portion 132 of analyte sensor 130 most susceptible to bending. In some embodiments, strain gauge 160 may be arranged at or adjacent to proximal end 131 of first portion 132 closest to skin surface S. However, in some embodiments, strain gauge 160 may be arranged at a midpoint of first portion 132 or at a distal end 133 of first portion 132 of analyte sensor 130.

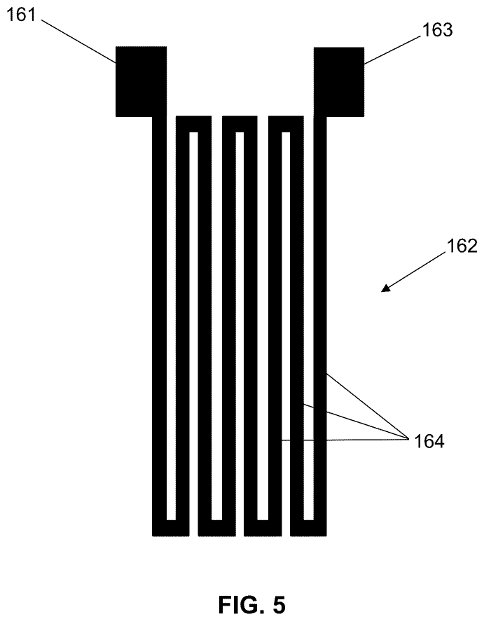

In some embodiments, strain gauge 160 includes a strain-sensitive resistor. Strain sensitive resistor may include a piezoresistor. In alternate embodiments, strain gauge 160 may include a piezoelectric strain gauge, among others. An exemplary piezoresistor is shown for example in FIG. 5. Piezoresistor may include an electrically conductive trace 162. The electrically conductive trace 162 may be referred to as a piezoresistive trace 162 or trace 162. Trace 162 may extend between a first electrical contact 161 and a second electrical contact 163, wherein the first and second electrical contacts 161, 163 are in electrical communication with sensor electronics. In some embodiments, trace 162 may have a serpentine pattern, and may include a plurality of closely-spaced, connected lines 164 that are substantially parallel to one another. Trace 162 may have a comb-like pattern.

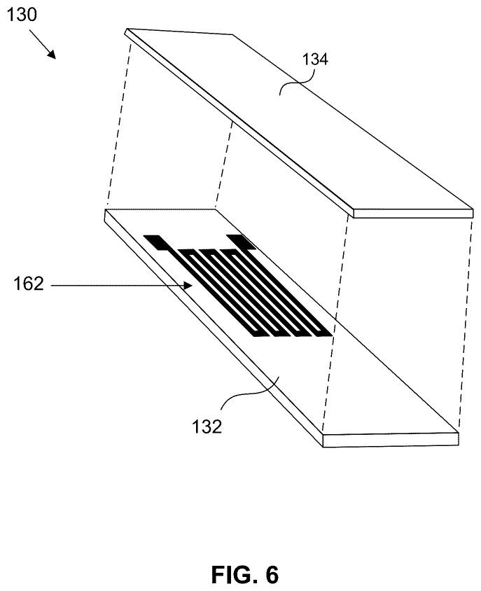

Trace 162 may be disposed on analyte sensor 130, as shown for example in FIG. 6. Trace 162 may be printed or etched onto a first portion 132 of analyte sensor 130 during manufacturing of analyte sensor 130. In some embodiments, trace 162 may be separately formed and may be coupled to substrate 132. Trace 162 may be arranged on first portion 132 such that lines 164 of trace 162 are arranged generally parallel to a longitudinal axis of first portion of analyte sensor 130. Piezoresistive trace 162 may be covered or encapsulated by one or more membranes 134 as described herein, such as a biocompatible membrane or mass transport limiting membrane. Piezoresistive trace 162 may be manufactured by manufacturing methods used to produce analyte sensor 130 such that the complexity of the manufacturing is not significantly increased due to inclusion of the strain gauge.

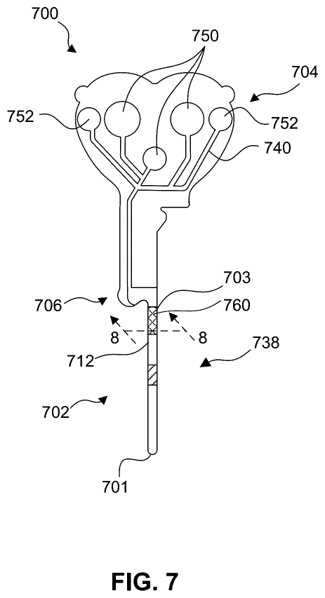

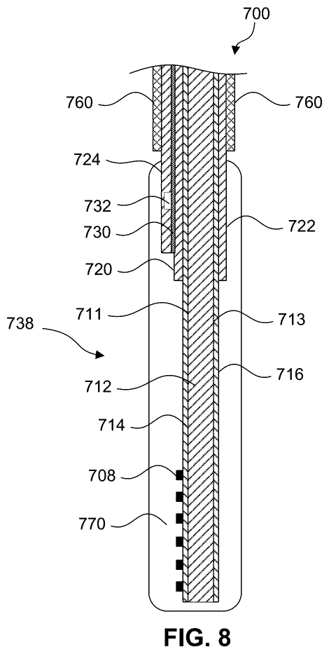

An analyte sensor 700 having first and second strain gauges 760 is shown, for example, in FIGS. 7 and 8. Analyte sensor 700 may include a first portion 702 configured to be inserted into a body of a user connected to a second portion 704 coupled to sensor electronics outside of a body of the user. First portion 702 may be connected to second portion 704 via an intermediate portion 706. Intermediate portion 706 may be configured to bend so that first portion 702 can be positioned at an angle relative to second portion 704, such as a 90 degree angle, when analyte sensor 700 is arranged in an analyte monitoring device. First portion 702 of analyte sensor 700 includes a distal or terminal end 701 and a proximal end 703 at which first portion 702 transitions to intermediate portion 706. First portion 702 includes sensing region 738 for detection of an analyte, such as glucose, among others. Second portion 704 may be enlarged relative to first portion 702. Second portion 704 may include electrical contacts 750 for electrical connection to sensor electronics. Electrodes of first portion 702 may be electrically connected to electrical contacts 750 via one or more electrical traces 740. Each electrode may have a corresponding electrical contact 750.

Analyte sensor 700 may include one or more strain gauges 760 as described herein. Strain gauge 760 may be positioned on first portion 702 of analyte sensor 700. Strain gauge 760 may be positioned outside of sensing region 738. Strain gauge 760 may be arranged at a proximal end 703 of first portion 702 of analyte sensor 700 adjacent intermediate portion 706. Strain gauge 760 may be arranged on a first side of substrate 712, an opposing second side of substrate 712, or a strain gauge 760 may be arranged on each side of substrate 712. Each strain gauge 760 is electrically connected to an electrical contact 752 via an electrical trace 740 for communicating data acquired by strain gauge 760 to the sensor electronics.

Analyte sensor 700 may include a layered construction as shown for example in FIG. 8. Analyte sensor 700 includes a substrate 712 which may be planar. Substrate 712 may include a non-conductive material. One or more electrodes are disposed on substrate 712, such as one or more of a working electrode, a counter electrode, and/or a reference electrode. In the embodiment of FIG. 8, a first side 711 of substrate 712 includes a working electrode 714, and an opposing second side 713 of substrate 712 includes a counter electrode 716. A sensing element 708, such as an analyte-responsive enzyme is arranged on working electrode 714. Sensing element 708 may be arranged discontinuously, such as in one or more discrete spots, as shown for example in FIG. 8.

Analyte sensor 700 further includes one or more dielectric layers. A first dielectric layer 720 is disposed on working electrode 714 and a second dielectric layer 722 is disposed on counter electrode 716. A reference electrode 730 is arranged on a surface of first dielectric layer 720, such that reference electrode 730 is electrically isolated from working electrode 714. Reference electrode 730 may include a reference element 732, such as silver (Ag) or silver chloride (AgCl) arranged on a surface of reference electrode 730. A third dielectric layer 724 may be disposed at least partially over reference electrode 730. Sensing region 738 including electrodes and sensing elements may be fully or partially encapsulated by one or more membranes 770 which may limit diffusion through the membrane, inhibit interferents, and/or promote biocompatibility.

A strain gauge 760 may be arranged on analyte sensor 700, and may be arranged outside of sensing region 738. Strain gauge 760 may be arranged on second dielectric layer 722, on third dielectric layer 724, or both. In some embodiments, for example, a first strain gauge 760 is arranged on second dielectric layer 722 and a second strain gauge 760 is arranged on third dielectric layer 724, as shown in FIG. 8, such that a pair of strain gauges 760 are arranged on opposing sides of substrate 712. In some embodiments, strain gauge 760 may be arranged on first dielectric layer 720. Strain gauge 760 may be electrically isolated from electrodes, such as by dielectric layers or by substrate 712.

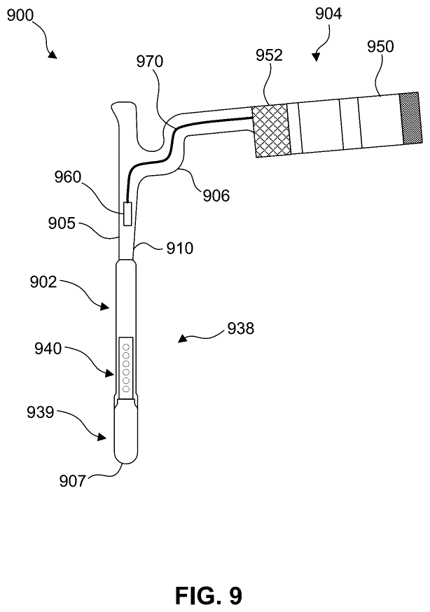

An analyte sensor 900 according to an embodiment is shown in FIG. 9. Similar to analyte sensor 700, analyte sensor 900 may include a first portion 902 configured to be inserted into a body of a user and that is connected to a second portion 904 configured to be coupled to sensor electronics outside of a body of the user. First portion 902 may be connected to second portion 904 via an intermediate portion 906. First portion 902 and second portion 904 may be coplanar. First portion 902 may be arranged at an angle, such as a 90 degree angle to second portion 904, such that analyte sensor 900 is generally L-shaped. Second portion 904 may include electrical contacts 950 for electrical connection to sensor electronics. Electrodes of first portion 902 may be electrically connected to electrical contacts 950 via electrical traces.

First portion 902 may include a sensing region 938 having one or more sensing areas. In FIG. 9, sensor 900 includes a first sensing area 939 and a second sensing area 940. First sensing area 939 may be arranged at distal end 907 of sensor 900, and second sensing area 940 may be arranged on analyte sensor 900 closer to proximal end 905 of first portion 902 so that first and second sensing areas 939, 940 are spaced longitudinally along analyte sensor 900. Each sensing area 939, 940 may include one or more electrodes and reagents for detection of an analyte, such as an analyte-responsive enzyme, among other components as discussed herein. First sensing area 939 may be configured to detect a first analyte, and second sensing area 940 may be configured to detect a second analyte that is different than the first analyte. In some embodiments, for example, first sensing area 939 may detect glucose, and second sensing area 940 may detect ketones, or vice versa.

A strain gauge 960 may be arranged on first portion 902 of analyte sensor 900. Strain gauge 960 may be arranged at proximal end 905 of first portion 902. Strain gauge 960 may be arranged outside of sensing region 938. Strain gauge 960 may be electrically connected to an electrical contact 952 on second portion 904 of analyte sensor 900, such as by an electrical trace 970, so that signals detected by strain gauge 960 can be communicated to sensor electronics, such as to a detection circuit as described herein. Strain gauge 960 may be arranged on a first side of analyte sensor 900, on an opposing second side, or on both sides, and may be arranged relative to electrodes in a layered construction, in a similar manner as described above with respect to FIG. 8.

An analyte sensor 1000 according to embodiments is shown in FIG. 10A. Analyte sensor 1000 has a cylindrical geometry. Analyte sensor 1000 may be configured as a wire. Analyte sensor 1000 includes one or more electrodes in a concentric or nested arrangement. As shown in FIG. 10A, analyte sensor 1000 includes a working electrode 1010. Working electrode 1010 may be a wire and may be an innermost portion of analyte sensor 1000. Working electrode 1010 may include a substrate with a conductive outer surface. Working electrode 1010 may include a metal, such as platinum, copper, titanium, or silver, among others. A dielectric layer 1020, also referred to as an insulating layer, may be arranged around working electrode 1010. Dielectric layer 1020 may have a generally tubular configuration. Dielectric layer 1020 may include polyurethane, among other materials. Dielectric layer 1020 may extend along a portion of working electrode 1010 between proximal and distal ends 1002, 1004, such that at least a portion 1012 of working electrode 1010 is exposed and is uncovered by dielectric layer 1020. The exposed portion of the working electrode 1010 may serve as a sensing region for detection of the analyte. A counter or reference electrode 1030 is arranged around at least a portion of dielectric layer 1020. The counter or reference electrode 1030 may include a reference element, such as silver (Ag) or silver chloride (AgCl) arranged on a surface of reference electrode. A second dielectric layer 1040 may surround the counter or reference electrode 1030. A strain gauge 1050 may be arranged on the second dielectric layer 1040. Strain gauge 1050 may be arranged at a proximal end 1004 of analyte sensor 1000. Electrodes 1010, 1030 and strain gauge 1050 may be electrically connected to one or more electrical contacts on a proximal portion of analyte sensor 1000 arranged above the skin surface of the user as described herein. In some embodiments, as shown in FIG. 10B, strain gauge 1050 may be arranged on dielectric layer 1020. In FIG. 10B, analyte sensor 1000 does not include a second dielectric layer 1040 as in FIG. 10A.

Analyte sensor 1000 may include one or more membranes at least partially covering a distal end 1002 of analyte sensor 1000. The one or more membranes may form a membrane assembly 1060. Membrane assembly 1060 may at least partially cover one or more of working electrode 1010, dielectric layer 1020, counter or reference electrode 1030, second dielectric layer 1040, or strain gauge 1050. In some embodiments, strain gauge 1050 may not be covered by membrane assembly 1060.

Membrane assembly 1060 may include one or more of an interferent layer, a sensing layer, and a resistance layer. Membrane assembly 1060 may be configured to facilitate detection of an analyte of interest. Membrane assembly 1060 may be configured to promote biocompatibility. Membrane assembly 1060 may be configured to limit diffusion of the analyte or interferents to the electrodes. In FIGS. 10A and 10B, analyte sensor 1000 includes an interferent layer 1062 configured to inhibit passage of interferents therethrough to prevent interferents from reaching working electrode 1010. Interferent layer 1062 may include alternating polyanionic and polycationic sublayers to minimize effects of electroactive interferents. A sensing layer 1064 may be arranged on the interferent layer 1062. Sensing layer 1064 may include sensing chemistry to facilitate detection of the analyte of interest as described herein. Membrane assembly 1060 may include a resistance layer 1066 arranged on the sensing layer 1064. Resistance layer 1066 may be configured to control or limit diffusion of glucose through membrane assembly 1060. In alternate embodiments, membrane assembly 1060 may include alternate or additional layers, and/or the layers may be arranged in a different order or sequence than described with respect to FIGS. 10A-10B.

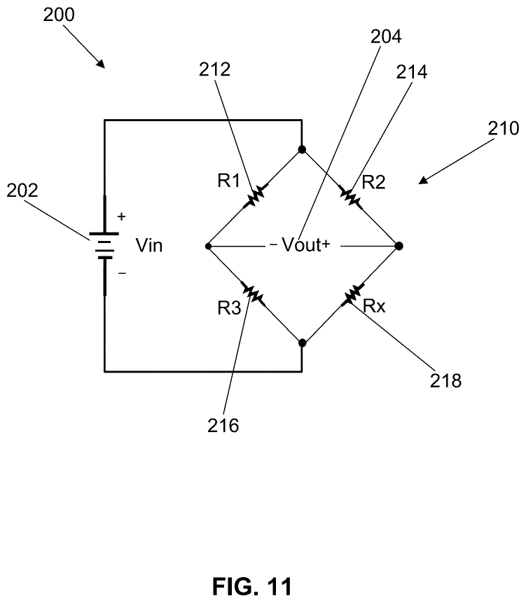

A detection circuit 200 for detecting bending of analyte sensor according to an embodiment is shown for example in FIG. 11. Detection circuit 200 may include a strain-sensitive resistor (e.g., a piezoresistor) as described herein. Detection circuit 200 may be configured to produce a voltage output that varies based on the application of strain to the strain-sensitive resistor 218.

In some embodiments, detection circuit 200 may include a Wheatstone bridge. Detection circuit 200 may include a voltage input 202 and a voltage output 204 and one or more resistors. The resistors may be arranged in a bridge 210. In the illustrated embodiment, detection circuit 200 includes four resistors arranged in two branches. However, it is understood that detection circuit 200 may include fewer or additional resistors and branches. In FIG. 11, first resistor 212, second resistor 214 and third resistor 216 each have a known resistance that is independent of application of strain. The fourth resistor is the strain-sensitive resistor 218 with a resistance that varies based on application of strain. In some embodiments, first, second, third, and fourth resistors 212, 214, 216, 218 may each have an electrical trace with the same pattern, and the strain-sensitive resistor 218 may be arranged on a portion of analyte sensor subject to strain whereas the remaining resistors may be positioned in an area not subject to strain. This may help to minimize or eliminate interference due to temperature or doping that may result if the resistors have different patterns or are formed by different processes.

When a voltage input is supplied to detection circuit 200, voltage output of the detection circuit 200 is zero when there is no strain on strain-sensitive resistor 218 and the R1, R3 branch of the bridge 210 is balanced with the R2, Rx branch of the bridge 210. However, when strain is applied to strain-sensitive resistor 218, the voltage output of detection circuit 200 is non-zero, indicating application of strain to strain-sensitive resistor 218. The voltage output of detection circuit 200 is proportional to the strain. The voltage output of detection circuit 200 may be a function of the change in length of the trace (e.g., trace 162) as discussed in further detail herein. The manner in which the resistance of strain-sensitive resistor 218 varies as a function of strain applied to the skin may depend on the materials and geometry of the strain gauge and analyte sensor.

A drive voltage may be supplied to voltage input 202 by sensor electronics 104 of analyte monitoring device 102 (see, e.g., FIG. 4). The drive voltage may be supplied substantially continuously to detection circuit 200 by sensor electronics 104, or may be supplied on a periodic basis to conserve energy. Further, a voltage at voltage output 204 may be detected by sensor electronics 104 of analyte monitoring device 102. In some embodiments, first, second and third resistors 212, 214, 216 are arranged within housing 110 as part of sensor electronics 104, and strain-sensitive resistor 218 is arranged on a first portion 132 of analyte sensor 130 external to housing 110. However, in some embodiments, one or more of the first, second and third resistors 212, 214, 216 may be arranged on first portion 132 of analyte sensor 130 in addition to strain-sensitive resistor 218.

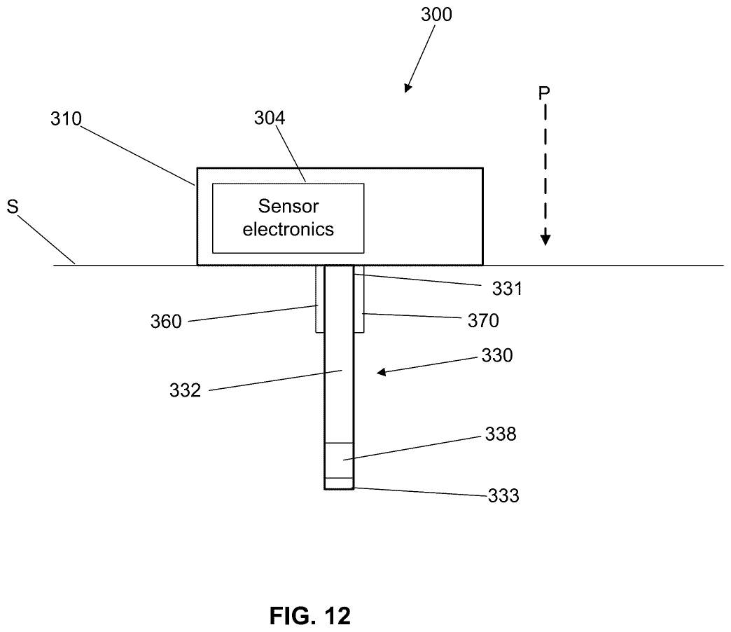

In some embodiments as shown in FIG. 12, analyte monitoring device 300 may include multiple strain gauges. Analyte monitoring device 300 is substantially the same as analyte monitoring device 102 and differs in including a second strain gauge. Thus, analyte monitoring device 300 includes sensor electronics 304 arranged within housing 310. A portion of analyte sensor 330 may also be arranged within housing 310. Analyte sensor 330 may be coupled to sensor electronics 304 such that signals are communicated to sensor electronics from analyte sensor 330. Analyte sensor 330 includes a first portion 332 having a proximal end 331 at housing 310 and a distal end 333 opposite proximal end 331 and extending away from housing 310. First portion 332 may include a sensing region 338 configured to be placed under a skin surface S to measure signals indicative of an analyte in a bodily fluid. Analyte monitoring device 300 includes a first strain gauge 360 and a second strain gauge 370 disposed on first portion 332 and configured to detect application of strain to first portion 332 of analyte sensor 330. In some embodiments, first strain gauge 360 is arranged on a first side of first portion 332 and second strain gauge 370 is arranged on an opposing, second side of first portion 332 (see, e.g., FIG. 8). As a result, when first strain gauge 360 is compressed, second strain gauge 370 is stretched, and vice versa.

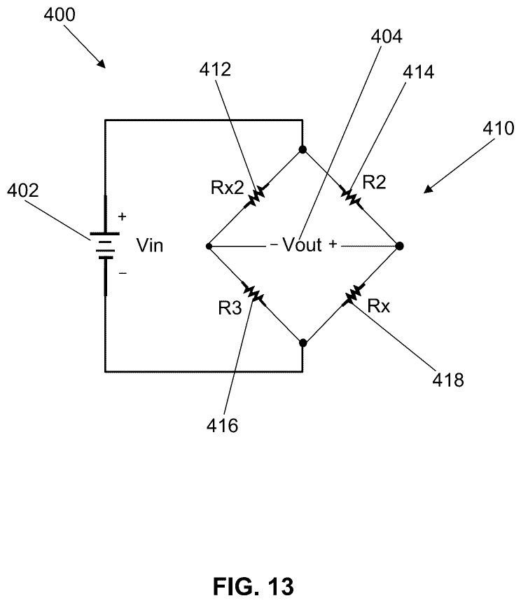

A detection circuit 400 for an analyte monitoring device having multiple strain-sensitive resistors is shown for example in FIG. 13. Detection circuit 400 is substantially the same as detection circuit 200, but differs in including a second strain-sensitive resistor, and the second strain sensitive resistor is in place of a resistor of known resistance. Thus, detection circuit includes a first resistor 414 and second resistor 416 having known resistance (that does not change in response to strain), and further includes a first strain-sensitive resistor 418 and a second strain-sensitive resistor 412. Second strain-sensitive resistor Rx2 may be arranged on different branch of bridge 410 than first strain-sensitive resistor Rx, such as in place of R1 of detection circuit 200. However, it is understood that the second strain-sensitive resistor could be placed in other locations, such as in the R3 position. As bending of analyte sensor is detected by each strain-sensitive resistor 412, 418, the application of strain to the analyte sensor results in a greater imbalance of the bridge 410 and a larger voltage output allowing for finer or more precise detection of strain on analyte sensor.

The voltage output of detection circuit 400 may be related to the change in length of the strain-sensitive resistor or resistors, such as the change in length of trace 162 shown in FIG. 5, by Equation 1 below:

$${V}_{out} = {V}_{in} ( \frac{Δ L}{2 L} )$$

wherein Vout is the voltage output, Vin is the voltage input, L is the unstretched length of the trace, and ΔL is the compressed/stretched length of the strain-sensitive resistor upon application of strain.

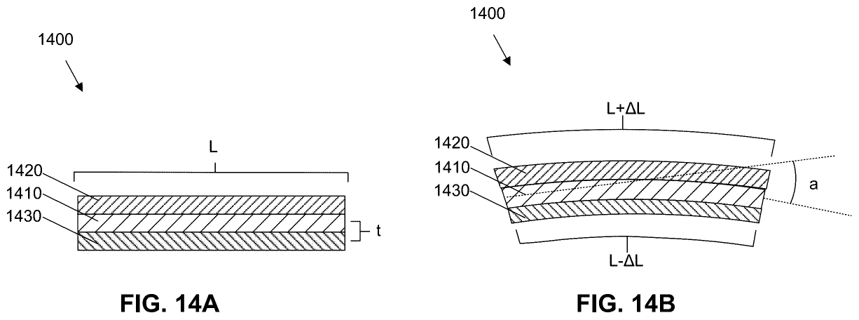

A portion of an analyte sensor 1400 having first strain-sensitive resistor 1420 and a second strain sensitive resistor 1430 on opposing sides of a substrate 1410, is shown in FIGS. 14A and 14B without and with strain applied to the analyte sensor 1400, respectively. In FIG. 14A, each strain-sensitive resistor 1420, 1430 has an unstretched length, L, measured in a direction of a longitudinal axis of substrate 1410, and a thickness, t, measured in a transverse direction of the longitudinal axis. In some embodiments, the length L may be in a range of 0.1 mm to 1 mm, and thickness t may be 0.05 mm to 0.25 mm. When strain is applied to analyte sensor 1400, analyte sensor 1400 may bend at an angle, a, as shown in FIG. 14B. As a result, first strain sensitive resistor 1420 may stretch to a length of L+ΔL and second strain sensitive resistor 1430 may compress to a length of L−ΔL. The change in length ΔL can be represented by Equation 2 below:

$${Δ}_{L = t * a}$$

wherein t is the strain-sensitive resistor thickness, and wherein the angle a is measured in radians. Bending of analyte sensor 1400 may result in a change in length of strain-sensitive resistor 1420, 1430 which results in a non-zero voltage output by the detection circuit, indicating application of strain to analyte sensor. In this way, the voltage output of the detection circuit may be proportional to the degree of bending and thus the applied strain, with a greater degree of bending corresponding to a greater applied pressure.

In some embodiments, an analyte level may be determined based at least in part on the detection of strain by the strain gauge. The analyte level may be determined by one or more processors in communication with the analyte monitoring device. The analyte level, determination of applied strain, and the analyte level alarms may be determined by the analyte monitoring device, the receiver device, a remote server, or a combination thereof. An analyte level may be determined based on an analyte signal detected by the analyte sensor, and the analyte level may be adjusted based on the strain detected by the strain gauge. As the applied pressure on the body may reduce blood flow, the application of pressure may artificially decrease the measured glucose level. The amount of pressure applied to the user's body, or the amount of bending of the analyte sensor may be correlated to an amount of error in the analyte level. This relationship of applied strain on the analyte sensor and the analyte level be based on a model, and may be improved over time such as by a machine learning algorithm. Thus, the analyte level may be corrected for the impact of the sensor strain. In some embodiments, the analyte level may be determined based in part on the strain only if the strain is at or above a predetermined threshold level.

In some embodiments, an analyte level alarm may be determined based on the analyte level and based on the strain detected by the strain gauge. The analyte level alarm may be output to a user by one or more processors in communication with the analyte monitoring device. The analyte level alarm may be produced, for example, by an audio element, such as a speaker, a vibration motor or haptic feedback device, a display screen, or by a combination thereof. The analyte level alarm may be output by a receiver device in communication with the analyte sensor. The receiver device may wirelessly communicate with the analyte monitoring device, such as by a Bluetooth communication protocol (e.g., BLE), and may include one or more processors for analyzing the received analyte data. The analyte monitoring device or the receiver device may determine whether the alarm condition is satisfied. The analyte monitoring device may determine that the alarm condition is satisfied and may send a signal to the receiver device to output the appropriate alarm. Alternatively, the receiver device may include software or programming to receive the analyte data and strain data from the analyte monitoring device and the receiver device may determine whether any alarm condition is satisfied.

An analyte level alarm may include a visual, auditory, vibratory alarm or a combination thereof. The visual alert may include display of an icon on a receiver device in communication with the analyte monitoring device. The visual alert may include display of a notification on the receiver device, such as a window, pop-up window, push notification, or the like. The notification may include an analyte level, rate of change of the analyte level, trend arrow, the analyte level threshold, or a combination thereof. The notification may be color-coded, and the color-coding may indicate a degree of severity of the alert. The notification may include text or a message explaining the alarm (e.g., high analyte level, low analyte level, urgent low analyte level, analyte level falling rapidly, or analyte level rising rapidly, etc.). The notification may include a tip or recommendation to help to guide the user's response. The alarm may include an audio alarm. The audio alarm may include a beep or tone. The alarm sound may be unique to each type of alarm, such that a low analyte level alarm has a first sound or tone, and a high analyte level alarm has a second, different sound or tone. The alarm sounds may include default settings, and may be customized by the user. The alarm may include voice or recorded speech. The alarm sound may reflect the degree of severity of the alarm, with more extreme analyte level alarms having louder sounds, higher pitch, or a longer duration, or a combination thereof. The alarm may repeat at a predetermined interval as the alarm condition persists. The alarm may repeat at a predetermined interval until the alarm is acknowledged by the user, such as by the user selecting an icon displayed on the receiver device to acknowledge the alarm.

Analyte level alarms include one or more low analyte level alarms, one or more high analyte level alarm, or one or more rate of change alarms, among others. The alarm condition for any of such alarms may require the analyte level to cross the analyte level threshold (e.g., the analyte level may cross a high analyte level threshold by exceeding the high analyte level threshold, or the analyte level may cross a low analyte level threshold by falling below the low analyte level threshold), and also that the strain detected by the strain gauge is 0 (or the strain is below a predetermined level) in order for the alarm condition to be satisfied and the alarm output to the user. This may help to prevent false alarms due to application of pressure on the user's body which may result in inaccurate analyte level measurements. For example, if a glucose level is determined to be below a low glucose threshold (e.g., 70 mg/dl) but strain is detected by the strain gauge, then the low glucose alarm may not be output to the user despite the presumed low glucose level.

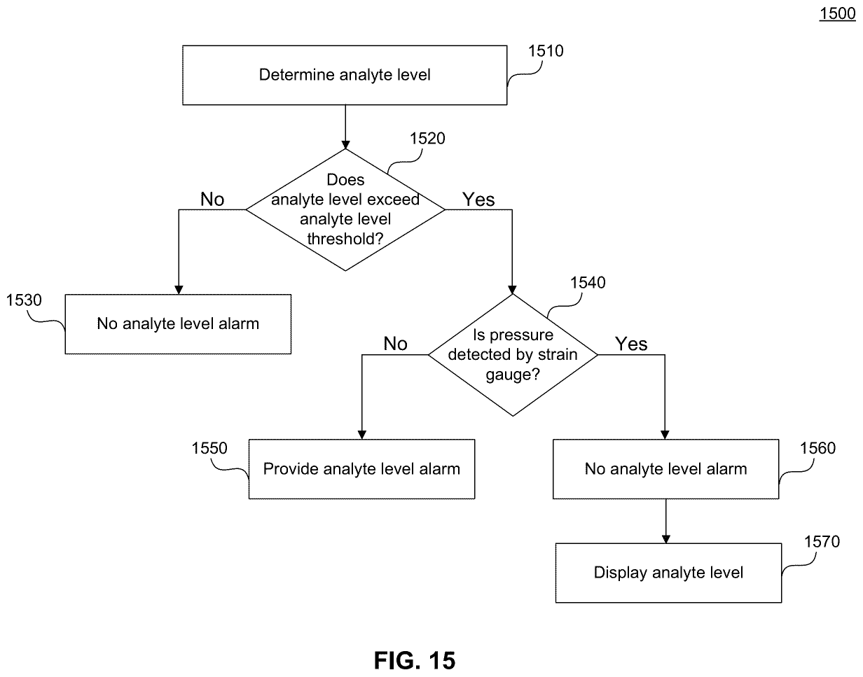

An exemplary method for providing an analyte level alarm 1500 is shown in FIG. 15. An analyte level is determined 1510 based on the signal from the analyte sensor. A comparison is made to determine if the analyte level crosses an analyte level threshold 1520, such as a low analyte level threshold. If the analyte level does not cross the threshold, no analyte level alarm is provided 1530. If the analyte level does cross the threshold, a determination is made whether strain is detected by the strain gauge 1540. If strain is not detected, the analyte level alarm is output 1550. If strain is detected, no alarm is output 1560 despite the analyte level crossing the analyte level threshold. This is because the strain may be indicative of an erroneous analyte level reading. When strain is detected, the analyte level may optionally be displayed to the user 1570 so that the user is informed of a potential high or low analyte level condition and take action if needed. The analyte level may be displayed along with a warning or notification that the analyte level may not be accurate.

The analyte level alarm condition is the condition that needs to be met for the analyte level alarm to be output. In some embodiments, an analyte level alarm condition may be adjusted when strain is detected. The alarm condition may be adjusted from a first alarm condition to a second alarm condition when strain is detected by the strain gauge, wherein the second alarm condition may be more stringent. The adjusted alarm condition may include a more extreme analyte level (e.g., a low analyte level threshold may be lowered, or a high analyte level threshold may be raised). For example, a low glucose threshold may be 70 mg/dl, but if strain is detected, the low glucose threshold may be lowered to 65 mg/dl. Alternatively, the alarm condition for a low glucose alarm may require a first number of glucose readings below the low analyte level threshold, e.g. below 70 mg/dl, and the adjusted alarm condition may require a second, greater number of glucose readings below the low analyte level threshold, e.g. below 70 mg/dl, in order for the low glucose alarm to be output. For example, under the original alarm condition, an alarm may be provided when a single analyte level is below the low analyte level threshold, e.g. below 70 mg/dl, and in the adjusted alarm condition, 5 consecutive readings must be below the low analyte level threshold, e.g. below 70 mg/dl, in order for the alarm to be provided, or an average of 10 consecutive readings must be below the low analyte level threshold, e.g. below 70 mg/dl, to satisfy the adjusted alarm condition. In some embodiments, the alarm condition for a low glucose alarm may be met as soon as a glucose reading below the low analyte level threshold, e.g. below 70 mg/dl, is measured, and the adjusted alarm condition may require the analyte level to remain below the low analyte level threshold, e.g. below 70 mg/dl, for a predetermined period of time (e.g., at least 5 minutes), thus delaying providing the low glucose alarm. This may help to ensure the glucose level is actually below the threshold and is not the result of applied strain. The adjusted alarm condition may also provide time to the user to move or adjust his or her position to relieve pressure applied to or around the analyte monitoring device.

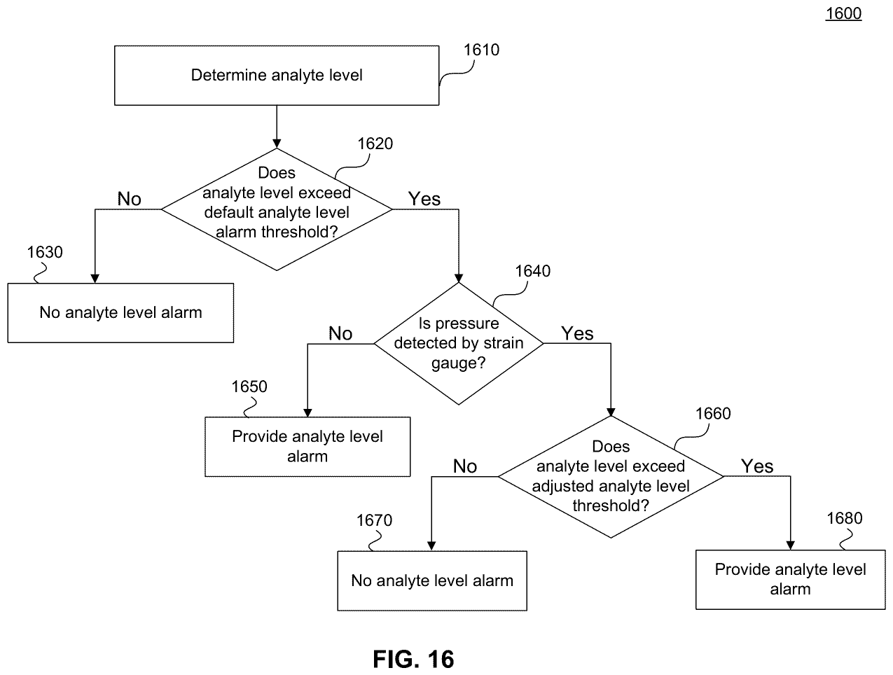

An exemplary method of providing an analyte level alarm 1600 is shown in FIG. 16. An analyte level is determined based on signals from analyte sensor 1610. The analyte level is compared to an analyte level threshold to determine if the analyte level crosses the threshold 1620. If the analyte level threshold is not crossed, no alarm is provided 1630. If the analyte level threshold is crossed, a determination is made whether strain is detected by strain gauge 1640. If strain is not detected, the analyte level alarm is provided 1650. If strain is detected, the alarm condition is adjusted and a determination is made whether the analyte levels meet the adjusted alarm condition 1660. If the adjusted alarm condition is not met, no analyte level alarm is output 1670, even though the analyte level may meet the original alarm condition. If the adjusted alarm condition is met, the alarm is then provided 1680.

In some embodiments, if strain is detected by the strain gauge, analyte level alarms may not be provided at all. The analyte level may still be output to the user so that the user can take action if needed. For example, if the threshold for a low glucose alarm is set to 70 mg/dl and the glucose level drops below 70 mg/dl, the glucose level may be output to the user, but the low glucose alarm may not be output due to the detection of strain by the strain gauge.

In some embodiments, if strain is detected, no analyte levels may be output to the user, no glucose alarms may be output, or both. As the strain may render the analyte readings inaccurate, no analyte levels or alarms may be output while the strain is detected. A notification may be provided to the user to notify the user that analyte levels or analyte level alarms are not provided.

In some embodiments, an alarm may be output to the user based on the detection of strain. For example, if strain is detected, or if strain above a predetermined level is detected, an alarm may be output to the user to notify the user of the detected strain. For example, the alarm may include a notification that a strain is detected. The notification may advise the user to change positions or otherwise attempt to remove the pressure. The notification may alternatively or additionally notify the user that no analyte levels and no analyte level alarms may be provided due to the strain.

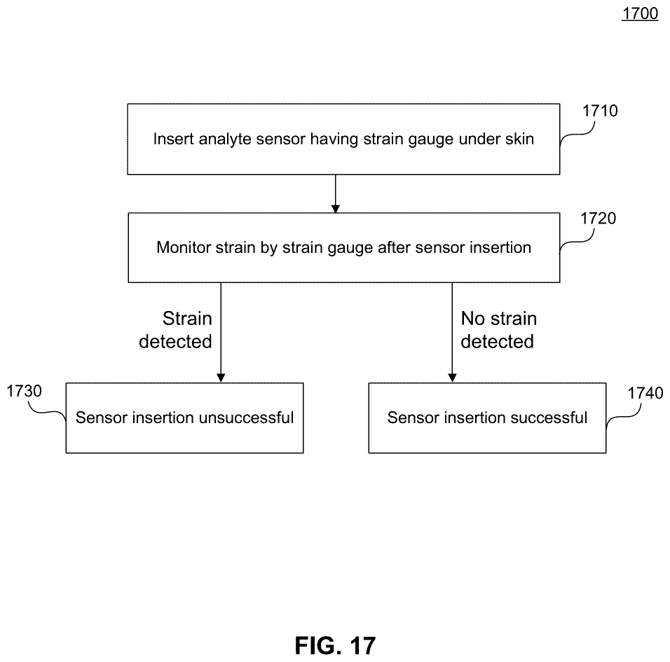

Some embodiments described herein relate to detecting successful insertion of an analyte monitoring device having a strain gauge into a user's body 1700, as shown in FIG. 17. An analyte monitoring device having a strain gauge may be inserted into the skin of a user 1710. A strain gauge of the analyte monitoring device detects strain 1720 after the analyte sensor is inserted into the user's body. The description of the analyte monitoring device and analyte monitoring system including the strain gauge above equally applies to this embodiment. If the strain is non-zero, a notification is provided that sensor insertion was not successful 1730. If the detected strain is zero, a notification is provided that sensor insertion was successful 1740. The analyte monitoring device may transmit an indication of successful or unsuccessful sensor insertion to a receiver device in wireless communication with the analyte monitoring device. The receiver device may output a notification indicative of successful or unsuccessful sensor insertion. When properly inserted, the analyte sensor should not be bent and thus the strain gauge should not detect any strain upon sensor insertion. If, however, strain is detected upon sensor insertion, this may indicate that the sensor insertion was not successful and the analyte sensor was bent during sensor insertion or is otherwise incorrectly positioned. This method of detecting success of sensor insertion may be beneficial in that it can provide immediate feedback to the user on the success of sensor insertion. Typically, the user waits until the sensor warms-up to determine if the sensor is measuring analyte levels correctly. As a result, the user may have to wait a period of time to confirm proper operation of sensor, and if the sensor is not functioning correctly the user may have to install a new sensor and again wait a period of time to confirm proper operation of the sensor.

In some embodiments, the method of detecting successful sensor insertion may further include applying a voltage to a detection circuit having a strain-sensitive resistor (e.g., detection circuit 200, 400) and measuring the output voltage after inserting the analyte sensor into the skin. If the output voltage of the detection circuit is non-zero, the sensor insertion is not successful, and if the output voltage is zero, the sensor insertion is successful.

It is to be appreciated that the Detailed Description section, and not the Summary and Abstract sections, is intended to be used to interpret the claims. The Summary and Abstract sections may set forth one or more but not all exemplary embodiments of the present invention(s) as contemplated by the inventors, and thus, are not intended to limit the present invention(s) and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention(s) that others can, by applying knowledge within the skill of the art, readily modify and/or adapt for various applications such specific embodiments, without undue experimentation, and without departing from the general concept of the present invention(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance herein.

The present invention can also be described in accordance with the following numbered clauses:

Clause 1. An analyte monitoring device, comprising:
a housing;
sensor electronics arranged within the housing; and
an analyte sensor comprising:
   a first portion configured to be placed under the skin surface of the user for measuring signals indicative of an analyte in a bodily fluid,
   a second portion configured to be arranged above the skin surface and that is coupled to the sensor electronics, and
   a strain gauge disposed on the first portion and configured to detect a strain applied to the first portion.

Clause 2. The analyte monitoring device of clause 1, wherein the strain gauge is a first strain gauge, wherein the first strain gauge is disposed on a first side of the first portion of the analyte sensor, and further comprising a second strain gauge disposed on a second side of the first portion opposite the first side of the first portion.

Clause 3. The analyte monitoring device of clause 1 or clause 2, wherein the strain gauge(s) comprises a strain-sensitive resistor.

Clause 4. The analyte monitoring device of clause 3, further comprising a detection circuit comprising a voltage input, a voltage output, and the strain-sensitive resistor.

Clause 5. The analyte monitoring device of clause 4, wherein the sensor electronics is configured to apply a drive voltage to the detection circuit and to detect a voltage output of the detection circuit.

Clause 6. The analyte monitoring device of any preceding clause, wherein the strain gauge(s) comprises a piezoresistive trace.

Clause 7. The analyte monitoring device of any preceding clause, wherein the first portion comprises a sensing region comprising one or more electrodes and an analyte-responsive reagent.

Clause 8. The analyte monitoring device of any preceding clause, wherein at least a portion of the strain gauge(s) is encapsulated by a membrane.

Clause 9. An analyte monitoring system, comprising:
an analyte monitoring device, comprising:
   an analyte sensor comprising a first portion configured to be placed under a skin surface of a user to detect signals indicative of analyte levels in a bodily fluid; and
   a strain gauge disposed on the first portion and configured to detect a strain applied to the first portion;
wherein the analyte monitoring system further comprises one or more processors in communication with the analyte monitoring device, and configured to:
   determine an analyte level based on the signals detected by the analyte sensor,
   determine a strain applied to the first portion detected by the strain gauge, and
   provide an analyte level alarm based on the analyte level and the strain detected by the strain gauge.

Clause 10. The analyte monitoring system of clause 9, wherein the analyte level alarm is output when the analyte level crosses an analyte level threshold and no strain is detected by the strain gauge.

Clause 11. The analyte monitoring system of clause 9 or clause 10, wherein the analyte level alarm is output when the analyte level crosses an analyte level threshold and the strain detected by the strain gauge is below a predetermined level.

Clause 12. The analyte monitoring system of any one of clauses 9 to 11, further comprising a receiver device in communication with the analyte monitoring device.

Clause 13. The analyte monitoring system of clause 12, wherein the receiver device comprises the one or more processors such that the receiver device is configured to provide the analyte level alarm.

Clause 14. The analyte monitoring system of any one of clauses 9 to 13, wherein the analyte level alarm comprises a first alarm condition, and wherein the analyte level alarm is adjusted from the first alarm condition to a second alarm condition when strain is detected by the strain gauge.

Clause 15. The analyte monitoring system of any one of clauses 9 to 14, wherein the first alarm condition comprises a first analyte level threshold, and wherein the second alarm condition comprises a second analyte level threshold that is different than the first analyte level threshold.

Clause 16. The analyte monitoring system of any one of clauses 9 to 14, wherein the first alarm condition is satisfied when a first number of analyte levels crosses an analyte level threshold, wherein the second alarm condition is satisfied when a second number of analyte levels cross the analyte level threshold, and wherein the second number is greater than the first number.

Clause 17. The analyte monitoring system of any one of clauses 9 to 14, wherein the first alarm condition is satisfied when an analyte level crosses an analyte level threshold, wherein the second alarm condition is satisfied when the analyte level exceeds the analyte level threshold for a predetermined period of time.

Clause 18. The analyte monitoring system of any one of clauses 9 to 14, wherein the first alarm condition is satisfied when an analyte level crosses an analyte level threshold, wherein the second alarm condition is satisfied when the analyte level is below the analyte level threshold for a predetermined period of time.

Clause 19. A method of determining successful insertion of an analyte sensor, the method comprising:
detecting a strain applied to an analyte sensor by a strain gauge disposed on the analyte sensor;
determining successful sensor insertion when no strain is detected after insertion of the sensor under the skin surface of the user;
determining unsuccessful sensor insertion when a strain is detected after insertion of the sensor under the skin surface of the user;
transmitting an indication of the successful sensor insertion or unsuccessful sensor insertion to a receiver device in wireless communication with the analyte sensor; and
outputting, by the receiver device, a notification indicative of successful sensor insertion or unsuccessful sensor insertion based on the indication.

Clause 20. The method of clause 19, wherein the strain gauge comprises a detection circuit comprising a strain-sensitive resistor, wherein the method further comprises applying a voltage to the detection circuit and detecting an output voltage, wherein a strain is detected when the output voltage is non-zero.

Clause 21. The method of clause 19 or clause 20, wherein the strain gauge comprises a piezoresistor.

Clause 22. An analyte monitoring device, comprising:
a housing;
sensor electronics arranged within the housing;
an analyte sensor at least partially arranged within the housing and coupled to the sensor electronics, wherein the analyte sensor comprises a first portion comprising a sensing region configured to be placed under a skin surface of a user, and a second portion configured to be placed above the skin surface and comprising one or more electrical contacts for communication with the sensor electronics; and
a strain gauge arranged on the first portion of the analyte sensor and electrically connected to an electrical contact of the one or more electrical contacts on the second portion of the analyte sensor by an electrical trace.

Clause 23. The analyte monitoring device of clause 22, wherein the sensing region comprises one or more electrodes arranged on a substrate of the analyte sensor.

Clause 24. The analyte monitoring device of clause 22 or clause 23, wherein the sensing region comprises a first sensing area for detecting a first analyte and a second sensing area for detecting a second analyte.

Clause 25. The analyte monitoring device of any of clauses 22 to 24, wherein the strain gauge is arranged on the first portion of the analyte sensor outside of the sensing region.

Clause 26. The analyte monitoring device of any of clauses 22 to 25, wherein the analyte sensor further comprises a substrate, a working electrode arranged on a first surface of the substrate and a counter electrode arranged on a second surface of the substrate, wherein the first and second surfaces are arranged on opposite sides of the substrate.

Clause 27. The analyte monitoring device of clause 26, wherein the analyte sensor further comprises a dielectric layer disposed on the working electrode, and a reference electrode disposed on the dielectric layer such that the reference electrode is electrically isolated from the working electrode.

Clause 28. The analyte monitoring device of clause 27, wherein a second dielectric layer is arranged on the reference electrode, and wherein the strain gauge is arranged on the second dielectric layer.

Clause 29. The analyte monitoring device of any of clauses 26 to 28, wherein the analyte sensor further comprises a dielectric layer arranged on the counter electrode, wherein the strain gauge is arranged on the dielectric layer.

Clause 30. The analyte monitoring device of any of clauses 22 to 29, wherein a membrane overcoats the sensing region of the analyte sensor.

Clause 31. A method of providing an analyte level alarm, the method comprising:
determining, by an analyte monitoring device comprising sensor electronics coupled to an analyte sensor, an analyte level based on the signals detected by the analyte sensor;
detecting, by a strain gauge coupled to the sensor electronics, a strain applied to the first portion; and
providing an analyte level alarm when an alarm condition is satisfied, wherein the alarm condition is based on the analyte level and the strain detected by the strain gauge.

Clause 32. The method of clause 31, wherein the analyte level alarm is provided when the analyte level crosses an analyte level threshold and no strain is detected by the strain gauge.

Clause 33. The method of clause 31, wherein the analyte level alarm is provided when the analyte level crosses an analyte level threshold and the strain detected by the strain gauge is below a predetermined level.

Clause 34. The method of any of clauses 31 to 33, further comprising communicating data from the analyte monitoring device to a receiver device, wherein the receiver device provides the analyte level alarm.

Clause 35. The method of clause 31, further comprising adjusting the alarm condition from a first alarm condition to a second alarm condition when strain is detected by the strain gauge.

Clause 36. The method of clause 35, wherein the first alarm condition comprises a first analyte level threshold, and wherein the second alarm condition comprises a second analyte level threshold that is different than the first analyte level threshold.

Clause 37. The method of clause 35, wherein the first alarm condition is satisfied when a first number of analyte levels crosses an analyte level threshold, wherein the second alarm condition is satisfied when a second number of analyte levels cross the analyte level threshold, and wherein the second number is greater than the first number.

Clause 38. The method of clause 35, wherein the first alarm condition is satisfied when an analyte level crosses an analyte level threshold, wherein the second alarm condition is satisfied when the analyte level exceeds the analyte level threshold for a predetermined period of time.

Clause 39. The method of clause 35, wherein the first alarm condition is satisfied when an analyte level crosses an analyte level threshold, wherein the second alarm condition is satisfied when the analyte level is below the analyte level threshold for a predetermined period of time.

Clause 40. A glucose monitoring system, comprising:
a glucose monitoring device, comprising:
a housing,
sensor electronics arranged within the housing,
a glucose sensor coupled to the housing and comprising a first portion configured to be placed under a skin surface of a user to detect signals indicative of glucose levels in a bodily fluid and a second portion coupled to the sensor electronics, and
a strain gauge disposed on the first portion of the glucose sensor and configured to detect strain applied to the first portion of the glucose sensor; and
one or more processors in communication with the glucose monitoring device, and configured to:
determine a glucose level based on the signals detected by the glucose sensor,
detect strain applied to the first portion by the strain gauge, and
output a glucose level alarm when an alarm condition is satisfied, wherein the alarm condition is based on the glucose level and the strain detected by the strain gauge.

Clause 41. The system of clause 40, wherein the alarm condition is satisfied when the glucose level crosses a glucose level threshold and no strain is detected by the strain gauge.

Clause 42. The system of clause 40, wherein the alarm condition is satisfied when the glucose level crosses a glucose level threshold and the strain detected by the strain gauge is below a predetermined level.

Clause 43. The system of any of clauses 40 to 42, further comprising a receiver device in wireless communication with the glucose monitoring device, wherein the receiver device is configured to output the glucose level alarm.

Clause 44. The system of any of clauses 40 to 43, wherein the alarm condition is a first alarm condition, and wherein the alarm condition is adjusted from the first alarm condition to a second alarm condition when strain is detected by the strain gauge.

Clause 45. The system of clause 44, wherein the first alarm condition comprises a first glucose level threshold, and wherein the second alarm condition comprises a second glucose level threshold that is different than the first glucose level threshold.

Clause 46. The system of clause 44, wherein the first alarm condition is satisfied when a first number of glucose levels crosses a glucose level threshold, wherein the second alarm condition is satisfied when a second number of glucose levels crosses the glucose level threshold, and wherein the second number is greater than the first number.

Clause 47. The system of clause 44, wherein the first alarm condition is satisfied when the glucose level exceeds a glucose level threshold, wherein the second alarm condition is satisfied when the glucose level exceeds the glucose level threshold for at least a predetermined period of time.

Clause 48. The system of any of clauses 40 to 47, wherein the strain gauge comprises a strain-sensitive resistor.

Clause 49. The system of clause 48, wherein the strain-sensitive resistor comprises a piezoresistive trace.

Clause 50. The system of clause 48, further comprising a detection circuit comprising a voltage input, a voltage output, and the strain-sensitive resistor.

Clause 51. The system of clause 40, wherein the sensor electronics is configured to apply a drive voltage to the detection circuit and to detect a voltage output of the detection circuit.

Clause 52. A method of providing a glucose level alarm, the method comprising:
detecting, by a glucose monitoring device, signals indicative of glucose levels in a bodily fluid, wherein the glucose monitoring device comprises a housing, sensor electronics arranged within the housing, a glucose sensor coupled to the housing and comprising a first portion placed under a skin surface of a user and a second portion coupled to the sensor electronics, and a strain gauge disposed on the first portion of the glucose sensor;
detecting, by the strain gauge of the glucose monitoring device, a strain applied to the first portion of the glucose sensor; and
outputting, by one or more processors in communication with the glucose monitoring device, a glucose level alarm when an alarm condition is satisfied, wherein the alarm condition is based on the glucose level and the strain detected by the strain gauge.

Clause 53. The method of clause 52, wherein the glucose level alarm is output when the glucose level crosses a glucose level threshold and no strain is detected by the strain gauge.

Clause 54. The method of clause 52, wherein the glucose level alarm is output when the glucose level crosses a glucose level threshold and the strain detected by the strain gauge is below a predetermined level.

Clause 55. The method of any of clauses 52 to 54, further comprising communicating data from the glucose monitoring device to a receiver device, wherein the receiver device outputs the glucose level alarm.

Clause 56. The method of any of clauses 52 to 55, further comprising adjusting the alarm condition from a first alarm condition to a second alarm condition when strain is detected by the strain gauge.

Clause 57. The method of clause 56, wherein the first alarm condition comprises a first glucose level threshold, and wherein the second alarm condition comprises a second glucose level threshold that is different than the first glucose level threshold.

Clause 58. The method of clause 56, wherein the first alarm condition is satisfied when a first number of glucose levels crosses a glucose level threshold, wherein the second alarm condition is satisfied when a second number of glucose levels crosses the glucose level threshold, and wherein the second number is greater than the first number.

Clause 59. The method of clause 56, wherein the first alarm condition is satisfied when a glucose level exceeds a glucose level threshold, wherein the second alarm condition is satisfied when the glucose level exceeds the glucose level threshold for at least a predetermined period of time.

What is claimed is:

1. A glucose monitoring system, comprising:
a glucose monitoring device, comprising:
a housing,
sensor electronics arranged within the housing,
a glucose sensor coupled to the housing and comprising a first portion configured to be placed under a skin surface of a user to detect signals indicative of glucose levels in a bodily fluid and a second portion coupled to the sensor electronics, and
a strain gauge disposed on the first portion of the glucose sensor and configured to detect strain applied to the first portion of the glucose sensor; and
one or more processors in communication with the glucose monitoring device, and configured to:
determine a glucose level based on the signals detected by the glucose sensor,
detect strain applied to the first portion by the strain gauge, and
output a glucose level alarm when an alarm condition is satisfied, wherein the alarm condition is based on the glucose level and the strain detected by the strain gauge.

2. The glucose monitoring system of claim 1, wherein the alarm condition is satisfied when the glucose level crosses a glucose level threshold and no strain is detected by the strain gauge.

3. The glucose monitoring system of claim 1, wherein the alarm condition is satisfied when the glucose level crosses a glucose level threshold and the strain detected by the strain gauge is below a predetermined level.

4. The glucose monitoring system of claim 1, further comprising a receiver device in wireless communication with the glucose monitoring device, wherein the receiver device is configured to output the glucose level alarm.

5. The glucose monitoring system of claim 1, wherein the alarm condition is a first alarm condition, and wherein the alarm condition is adjusted from the first alarm condition to a second alarm condition when strain is detected by the strain gauge.

6. The glucose monitoring system of claim 5, wherein the first alarm condition comprises a first glucose level threshold, and wherein the second alarm condition comprises a second glucose level threshold that is different than the first glucose level threshold.

7. The glucose monitoring system of claim 5, wherein the first alarm condition is satisfied when a first number of glucose levels crosses a glucose level threshold, wherein the second alarm condition is satisfied when a second number of glucose levels crosses the glucose level threshold, and wherein the second number is greater than the first number.

8. The glucose monitoring system of claim 5, wherein the first alarm condition is satisfied when the glucose level exceeds a glucose level threshold, wherein the second alarm condition is satisfied when the glucose level exceeds the glucose level threshold for at least a predetermined period of time.

9. The glucose monitoring device of claim 1, wherein the strain gauge comprises a strain-sensitive resistor.

10. The glucose monitoring device of claim 9, wherein the strain-sensitive resistor comprises a piezoresistive trace.

11. The glucose monitoring device of claim 9, further comprising a detection circuit comprising a voltage input, a voltage output, and the strain-sensitive resistor.

12. The analyte monitoring device of claim 11, wherein the sensor electronics is configured to apply a drive voltage to the detection circuit and to detect a voltage output of the detection circuit.

13. A method of providing a glucose level alarm, the method comprising:
detecting, by a glucose monitoring device, signals indicative of glucose levels in a bodily fluid, wherein the glucose monitoring device comprises a housing, sensor electronics arranged within the housing, a glucose sensor coupled to the housing and comprising a first portion placed under a skin surface of a user and a second portion coupled to the sensor electronics, and a strain gauge disposed on the first portion of the glucose sensor;
detecting, by the strain gauge of the glucose monitoring device, a strain applied to the first portion of the glucose sensor; and
outputting, by one or more processors in communication with the glucose monitoring device, a glucose level alarm when an alarm condition is satisfied, wherein the alarm condition is based on the glucose level and the strain detected by the strain gauge.

14. The method of claim 13, wherein the glucose level alarm is output when the glucose level crosses a glucose level threshold and no strain is detected by the strain gauge.

15. The method of claim 13, wherein the glucose level alarm is output when the glucose level crosses a glucose level threshold and the strain detected by the strain gauge is below a predetermined level.

16. The method of claim 13, further comprising communicating data from the glucose monitoring device to a receiver device, wherein the receiver device outputs the glucose level alarm.

17. The method of claim 13, further comprising adjusting the alarm condition from a first alarm condition to a second alarm condition when strain is detected by the strain gauge.

18. The method of claim 17, wherein the first alarm condition comprises a first glucose level threshold, and wherein the second alarm condition comprises a second glucose level threshold that is different than the first glucose level threshold.

19. The method of claim 17, wherein the first alarm condition is satisfied when a first number of glucose levels crosses a glucose level threshold, wherein the second alarm condition is satisfied when a second number of glucose levels crosses the glucose level threshold, and wherein the second number is greater than the first number.

20. The method of claim 17, wherein the first alarm condition is satisfied when a glucose level exceeds a glucose level threshold, wherein the second alarm condition is satisfied when the glucose level exceeds the glucose level threshold for at least a predetermined period of time.
